(19) United States
(12) Patent Application Publication
(10) Pub. No.: US 2008/0139884 A1
(43) Pub. Date: Jun. 12, 2008

(54) MEDICAL EXAMINATION SYSTEM WITH ENDOSCOPIC PROBE

(76) Inventor: William D. Myers, Birmingham, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/567,583

(22) Filed: Dec. 6, 2006

(51) Int. Cl. A61B 1/01

(52) U.S. Cl. 600/118

(57) ABSTRACT

A medical examination system having an endoscopic probe with two ends and adapted to be ingested in the gastrointestinal tract of the body. A first force field is applied to one end of the probe and a second and opposite force field is applied to the other end of the probe. A manipulator then applies at least one of the force fields exteriorly of the body to the probe to thereby manipulate the position and/or orientation of the probe.

### BACKGROUND OF THE INVENTION

I. Field of the Invention

The present invention relates generally to a medical examination system utilizing an endoscopic probe.

II. Description of Related Art

Endoscopic probes have been recently developed for inspecting the gastrointestinal tract of a living organism, such as a human. Such probes are typically ingested and travel through the gastrointestinal tract in the normal fashion until they are ultimately expelled from the body.

Such previously known endoscopic probes typically contain a camera and/or other sensors. The camera and/or the other sensors take pictures or measurements of the gastrointestinal tract as the endoscopic probe passes through the tract. The data collected by the endoscopic probe may be either collected in random access memory inside the probe itself, or transmitted by the probe via radio signal to an appropriate receiver outside the body.

while these previously known endoscopic probes enjoy significant advantages over other previously known methods of examining the gastrointestinal tract of a living organism, such endoscopic probes suffer from several disadvantages. One such disadvantage is that the probe may become lodged within the gastrointestinal tract so that it does not pass naturally from the body. In such cases, drastic measures, including surgery, may be required in order to retrieve the probe.

A still further disadvantage of the previously known endoscopic probes is that the probe typically takes a picture or other sensor reading in only a single direction with respect to the probe itself. Consequently, in the event that the probe is oriented in the wrong direction as the probe passes across an area of interest in the gastrointestinal tract, the probe may fail to take sufficient data or indeed any data at all of the area of interest in the gastrointestinal tract of the patient. When this occurs, it may be necessary to repeat the entire procedure with a new endoscopic probe.

Similarly, in some cases it would be desirable to slow or even halt the travel of the endoscopic probe through the gastrointestinal tract in order to take multiple sensor readings or pictures of the area of interest. However, since the previously known endoscopic probes pass through the gastrointestinal tract at an uncontrolled speed, insufficient data may be collected

### SUMMARY OF THE INVENTION

The present invention provides a system for endoscopic inspection of the gastrointestinal tract of a living organism which overcomes the above-mentioned disadvantages of the previously known devices.

In brief the system of the present invention comprises an endoscopic probe having two ends and which is adapted to be ingested by a living organism, such as a human. The probe contains a housing and includes interior sensors within the housing which collect data as the endoscopic probe passes through the gastrointestinal tract of the body.

A first force field, such as a magnetic pole, is applied to one end of the probe. Similarly, a second force field, such as a magnetic pole, is applied to the other end of the probe. The first and second force fields, furthermore, are opposite from each other.

A manipulator then applies at least one of the force fields exteriorly of the body to the probe to thereby manipulate the position and/or orientation of the probe. Since the position and/or orientation of the probe is controlled by the manipulator, the probe may be properly positioned and even held in place by medical personnel in order to assure that sufficient data is collected from areas of interest in the gastrointestinal tract of the body.

Although preferably opposite magnetic poles are applied to opposite ends of the probe, other force fields may alternatively be used. For example, a positive electrostatic force field may be applied to one end of the probe and the opposite charge static field applied to the other. Still other types of fields may be used without deviating from the spirit or scope of the invention.

### BRIEF DESCRIPTION OF THE DRAWING

A better understanding of the present invention will be had upon reference to the following detailed description wherein lice reference characters refer to like parts throughout the several views and in which:

FIG. 1 is a diagrammatic view illustrating a preferred embodiment of the system of the present invention;

FIG. 2 is an elevational view illustrating an exemplary endoscopic probe; and

FIG. 3 is a plan view illustrating the probe of the present invention; and

FIG. 4 is a view similar to FIG. 3, but illustrating the operation of the present invention.

### DETAILED DESCRIPTION OF A PREFERRED EMBODIMENT OF THE PRESENT INVENTION

With reference first to FIGS. 2 and 3, an endoscopic probe 10 is shown and which is dimensioned to be ingested orally into the mouth of a living organism, such as a human. The probe 10 includes an outer housing 12 which defines an interior 14 in which one or more sensors 16 are contained. The sensors 16 may comprise an optical or infrared camera or other types of sensors. In many cases, the sensors 16 take data in a predetermined direction relative to the probe body 12, e.g. through a window 18 on the probe body 12.

Referring to FIG. 3, a first force field 20 is applied to one end of the probe body 12 while, similarly, a second force field 22 opposite from the first force field 20 is applied to the opposite end of the body 12. The force fields 20 and 22, furthermore, may take any of several configurations.

For example, a ferrous coating may be applied to both ends of the probe body 12 and then magnetized. In this case, one magnetic pole, e.g. a south magnetic pole, forms the first force field 20 while the north magnetic pole would form the second force field 22.

Alternatively, the force fields 20 and 22 may comprise electric force fields. Such electric force fields may be created by applying a positive electrostatic charge to one end of the capsule 12 and the opposite electrostatic charge to the opposite end of the capsule body 12. Still other types of force fields may be applied without deviation from the spirit or scope of the invention.

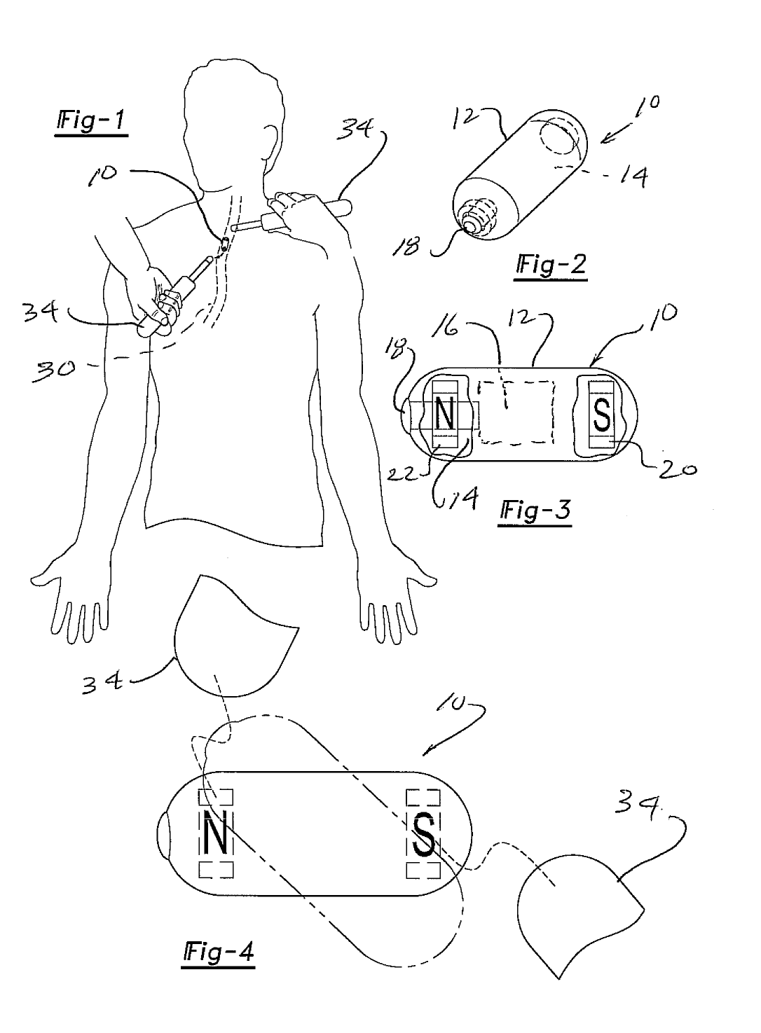

With reference now to FIG. 1, in use the endoscopic probe 10 is introduced into the gastrointestinal tract 30 of a living organism 32, such as a human. Preferably, the probe 10 is introduced into the tract 30 by the patient simply ingesting the probe 10. The probe 10 will then proceed to travel through the gastrointestinal tract 30 in the normal fashion.

With reference to FIGS. 1 and 4, in order to control the position and/or orientation of the probe 10 as it passes through the gastrointestinal tract 30, one or more manipulators 34 are provided. Each manipulator 34 (only one illustrated in FIG. 2) imposes a first force field on the probe 12 from a position exterior of the body 32. For example, assuming that the force fields 20 and 22 each comprise opposite magnetic fields, the manipulator 34 comprises a magnet. Consequently, by positioning the magnet exteriorly of the body 32, but relatively close to the probe 10, the magnetic field from the manipulator 34 coacts with the magnetic field from the force fields 20 and 22 on the probe 10 in order to mechanically position and/or orient the probe 10 in the gastrointestinal tract 30 as a function of the position of the manipulator 34 as shown in phantom line in FIG. 4.

The manipulator 34 may also comprise an electromagnet powered by a control source 36. In such a situation, the power of the electromagnet may be adjusted by the control 36 in order to more accurately control and orient the position of the probe 10.

Similarly, in the event that the force fields 20 and 22 on the probe 10 comprise electric fields, the manipulator 34 would impart an electric field on the probe 10 in the gastrointestinal tract 30 as a function of the position of the manipulator 34.

In practice, by manipulating both the position and orientation of the probe 10 within the gastrointestinal tract 303 medical personnel may control the amount and type of data acquired by the probe 10. Furthermore, since the probe 10 preferably transmits a radio signal of the probe sensor data exteriorly of the body 32, the positioning of the probe 10 by the medical personnel utilizing the manipulators 34 and data acquisition may be controlled on a real-time basis.

From the foregoing, it can be seen that the present invention provides a simple and yet effective medical examination system for the gastrointestinal tract. Having described my invention, however, many modifications thereto will become apparent to those skilled in the art to which it pertains without deviation from the spirit of the invention as defined by the scope of the appended claims.

I claim:

1. A medical examination system comprising:
an endoscopic probe having two ends and adapted to be inserted into the gastrointestinal tract of a body,
a first force field applied to one end of said probe and a second force field applied to the other end of said probe, said first and second force fields being opposite from each other,
a manipulator which applies at least one of said force fields exteriorly of the body to thereby manipulate the position and/or orientation of said probe.

2. The invention as defined in claim 1 wherein said first force field comprises a first magnetic pole and wherein said second force field comprises a second magnetic pole opposite from said first magnetic field.

3. The invention as defined in claim 2 wherein said manipulator comprises a magnet.

4. The invention as defined in claim 3 wherein said manipulator comprises an electromagnet.

5. The invention as defined in claim 1 wherein said probe includes a camera.

6. The invention as defined in claim 1 wherein one of said force fields comprises a positive electrostatic charge and the other force field comprises a negative electrostatic charge.

7. The invention as defined in claim 6 wherein said manipulator comprises means for creating an electric field.
